(19) United States
(12) Patent Application Publication
(10) Pub. No.: US 2014/0114214 A1
(43) Pub. Date: Apr. 24, 2014

(54) GAIT ASYMMETRY MEASUREMENT

(71) Applicants: Patrick Esser, Simpelveld (NL); Ken Howells, Oxfordshire (GB); Helen Dawes, Oxfordshire (GB); Johnny Collet, Oxfordshire (GB)

(72) Inventors: Patrick Esser, Simpelveld (NL); Ken Howells, Oxfordshire (GB); Helen Dawes, Oxfordshire (GB); Johnny Collet, Oxfordshire (GB)

(73) Assignee: OXFORD BROOKES UNIVERSITY, Oxfordshire (GB)

(21) Appl. No.: 14/122,344

(22) PCT Filed: May 25, 2012
(86) PCT No.: PCT/GB2012/051181

§ 371 Date: Dec. 10, 2013

(30) Foreign Application Priority Data
May 27, 2011 (GB) 1108952.1

(51) Int. Cl. A61B 5/11; A61B 5/00

(52) U.S. Cl. CPC A61B 5/112; A61B 5/742; A61B 5/72
USPC 600/595

(57) ABSTRACT

A system for measuring variation in the gait of a subject comprises a sensor arranged to measure variations in vertical position of the subject while the subject takes a series of steps, a processor, and a display. The processor is arranged to identify a plurality of points in a first one of the steps and a plurality of points in a second one of the steps, to identify a plurality of pairs of the points, each pair comprising one point in each of the steps, to determine a value of height for each of the points in each of the pairs, and to control the display to produce a display plotting the heights of the two points in each pair against each other.

### FIELD OF THE INVENTION

The present invention relates to the measurement of gait and in particular to the measurement of asymmetry in gait.

### BACKGROUND TO THE INVENTION

Variability is always present in human movement. Commonly variability is seen as noise, thus is unwanted and removed by data processing. Variability analysis has been used to indentify human gait patterns, but also for clinical purposes such as analysing pathological gait. Studies have found increased step time variability in elderly subjects compared to young adults. Increase in stride-to-stride variability within elderly subjects has also been found.

Parkinson's disease (PD), a progressive disorder of the central nervous system, presents with resting tremor, short slow steps, decreased centre of mass (CoM) movement and an increase in variability of temporospatial parameters of gait such as stride length and step time. Research has also shown that people with idiopathic Parkinson's disease have a higher gait asymmetry compared to age matched controls. Studies have also been carried out which suggest that gait asymmetry is common when looking at temporospatial parameters in both typically developed adults (TDA) and PD. In these studies fractal analysis has been used, which relies on longer walks (i.e. 2 and 6 minute walking tests). An issue with these studies is that the data sets required are relatively large. Gathering this amount of data can be seen as time consuming and therefore rather stressful for the participant, especially those with clinical conditions.

As described, for example, in WO2010/073044 inertial measurement unit (IMU) technology can be used to measure centre of mass (CoM) movement, providing a quick and relatively cheap way of gathering larger amounts of data over relative few steps where a high sample frequency is used.

### SUMMARY OF THE INVENTION

The present invention provides a system for measuring variation in the gait of a subject, the system comprising measuring means arranged to measure variations in the position of the subject while the subject takes a series of steps. The position may be a vertical position, but may be position in any direction, or along any axis. The direction or axis may be close to vertical, or may be a horizontal direction or axis. The system may further comprise processing means. The system may further comprise display means. The processing means may be arranged to identify a plurality of points in a first one of the steps and a plurality of points in a second one of the steps. The processing means may be arranged to identify a plurality of pairs of the points. Each pair may comprise one point in each of the steps. The processing means may be arranged to determine a value of height for each of the points in each of the pairs. The processing means may be arranged to control the display means to produce a display plotting the heights of the two points in each pair against each other.

The processing means may be arranged to identify the first and second steps as consecutive steps in the series. However, the first and second steps may be selected from anywhere in the series. Where the system is arranged to measure asymmetry between the legs of a subject, the first and second steps may be selected to be on different legs. However in some cases they may be different steps on the same leg where variation over time between steps on the same leg is being measured.

The processing means may be arranged to identify each pair of points so that the time interval between the two points in each pair is the same. Alternatively the time interval may be different for different pairs of points. There may be some other relationship between the pairs of points, such as their relative position within the step cycle.

The processing means may be arranged to identify a series of points through the series of steps and to include each of the series of points in one of the pairs. This allows the use of a constant (or regularly varying) sampling interval throughout the test walking period.

The processing means may be arranged to define a coordinate system having two axes representing the heights of the two points in a pair. This can allow the heights of each pair of points to be represented by a position within the coordinate system. The processing means may be arranged to identify the positions in the coordinate system for each of the pairs of points. The processing means may be arranged to control the display so as to indicate those positions, for example by means of respective markers. The markers may comprise dots or crosses or be of any other suitable shape.

The processing means may be arranged to analyse the positions associated with the pairs of points and to calculate a parameter of the positions. The parameter may be a statistical parameter, for example a standard deviation of distance from a point or a line, or it may be an average position.

The processing means may be arranged to calculate the position of a line having a predetermined relationship to the positions associated with the pairs of the points. The line may be a straight line, or it may be a curve. For example it may be an oval, or a circle. The processing means may be arranged to control the display to display the line.

The present invention further provides a method of measuring variation in the gait of a subject. The method includes measuring variations in vertical position of the subject while the subject takes a series of steps. The method may comprise any one or more of: identifying a plurality of points in a first one of the steps and a plurality of points in a second one of the steps; identifying a plurality of pairs of the points, each pair comprising one point in each of the steps; determining a value of height for each of the points in each of the pairs; and, producing a display plotting the heights of the two points in each pair against each other.

The first and second steps may be identified as consecutive steps in the series. The pairs of points may be identified so that the time interval between the two points in each pair is the same.

The method may include identifying a series of points through the series of steps and including each of the series of points in one of the pairs.

The method may include defining a coordinate system having two axes representing the heights of the two points in a pair, so that the heights of each pair of points can be represented by a position within the coordinate system.

The method may include identifying the positions in the coordinate system for each of the pairs of points, and controlling the display so as to indicate those positions.

The method may include analysing the positions associated with the pairs of points and calculating a parameter of the positions.

The method may include calculating the position of a line having a predetermined relationship to the positions of the points, and controlling the display to display the line.

The present invention further provides a system for measuring variation in the gait of a subject, the system comprising a memory arranged to store data recording variations in vertical position of the subject while the subject takes a series of steps, processing means, and display means. The processing means may be arranged to identify a plurality of points in a first one of the steps and a plurality of points in a second one of the steps. The processing means may be arranged to identify a plurality of pairs of the points, each pair comprising one point in each of the steps. The processing means may be arranged to determine a value of height for each of the points in each of the pairs. The processing means may be arranged to control the display means to produce a display plotting the heights of the two points in each pair against each other.

Preferred embodiments of the present invention will now be described by way of example only with reference to the accompanying drawings.

### BRIEF DESCRIPTION OF THE DRAWINGS

FIG. 1 is a diagram of a subject with an inertial measurement unit used in an embodiment of the invention;

FIG. 2 is a diagram of a computer system according to an embodiment of the invention arranged to analyse data from the IMU of FIG. 1;

FIG. 3 is a computer simulated plot of height of the CoM of the subject of FIG. 1 as a function of time with variation in step length;

FIG. 4 shows the plot of FIG. 3 superimposed on a copy of the same plot shifted in time by the duration of one step;

FIG. 5 is a plot of CoM excursion from the shifted plot as a function of the CoM excursion from the original plot, with values for each step on the horizontal axis plotted against values for the previous step on the vertical axis;

FIG. 6 is a plot of measured height of the CoM of the subject of FIG. 1 during walking as a function of time;

FIG. 7 is a plot similar to FIG. 5 for the data of FIG. 6;

FIG. 8 is a diagram showing parameters of the plot of FIG. 7 which can be quantified in an embodiment of the invention;

FIGS. 9a-9h are computer simulated plots similar to that of FIG. 5 for sine waves of equal amplitude and wavelength, but out of phase by 180° (a), 225° (b), 270° (c), 315° (d), 360° (e), 45° (f), 90° (g) and 135° (h);

FIGS. 10a-d are computer simulated plots similar to that of FIG. 5 for two sine waves (frequency 10.1 Hz, amplitude 5 and 7 cm) being out of phase by 180° (a), 170° (b) 360° (c) and 90° (d);

FIGS. 11a and 11b are computer simulated plots based on three sine waves (constant amplitude of 5 cm and phase shift of)180° representing a change in step length (a) and step frequency (b); and

FIG. 12 is a set of three plots similar to that of FIG. 5 for PD sufferers and three plots for typically developed adults generated from measured data.

### DESCRIPTION OF THE PREFERRED EMBODIMENTS

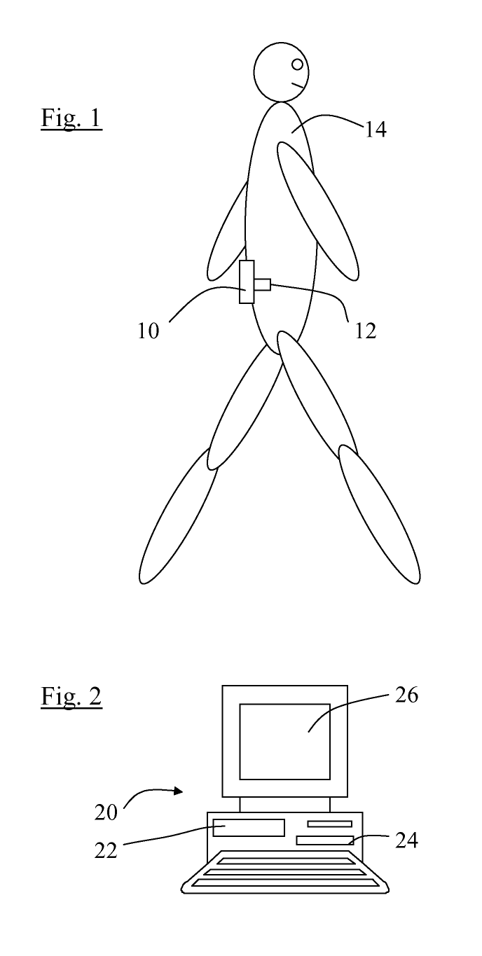

Referring to FIG. 1 an inertial measurement unit (IMU) 10 is attached to a subject 14 by means of adhesive tape 12. The IMU is of a known form and comprises a number of accelerometers and is arranged to identify the vertical direction using the accelerometers and to measure acceleration in the vertical direction. It is then arranged to calculate vertical velocity and hence vertical position continuously and to store the calculated vertical position at a series of sample points in time. The vertical position can be relative to any reference position or height, which in this case is the position of the CoM at the start of measurement when the subject is standing still. In this embodiment the measured vertical position is measured and defined by the IMU 10 as a CoM excursion, which is the difference between the actual vertical position of the CoM of the subject at a point in time and the reference, starting vertical position of the CoM.

Referring to FIG. 2 a computer system 20 is arranged to receive the CoM excursion data collected over a test period and analyse it and use it to generate various displays which are arranged to help with the identification of various characteristics of the gait of the subject. The computer system comprises a processor 22, a memory 24 and a display screen 26. In conventional manner the memory is arranged to store the data collected by the IMU 10, and also to store a program which controls the processing of the data by the processor 22 and controls the display screen 26. It will be appreciated that the amount of processing of the raw sensor signals that is carried out in the IMU 10 and the amount that is done by the computer system 20 is arbitrary.

The CoM movement of the subject can be described using an inverted pendulum model of the gait of the subject. This model describes the mechanical energetic state during a gait cycle in which the CoM excursion plotted as a function of time behaves like a sine wave having a sequence of peaks and troughs, with each step producing one cycle of the sine wave from the bottom of one trough to the bottom of the next. It will be appreciated that any height measure will give the same basic shape, with the reference height relative to which the height is measured being to some extent arbitrary. In this embodiment of the invention further analysis using a non-linear method is performed on the CoM excursion data by the computer system 20, whereby CoM Excursion (CoM Excursionᵢ) at each point in one step is plotted against the CoM Excursion (CoM Excursionᵢ) for the same point in the previous step.

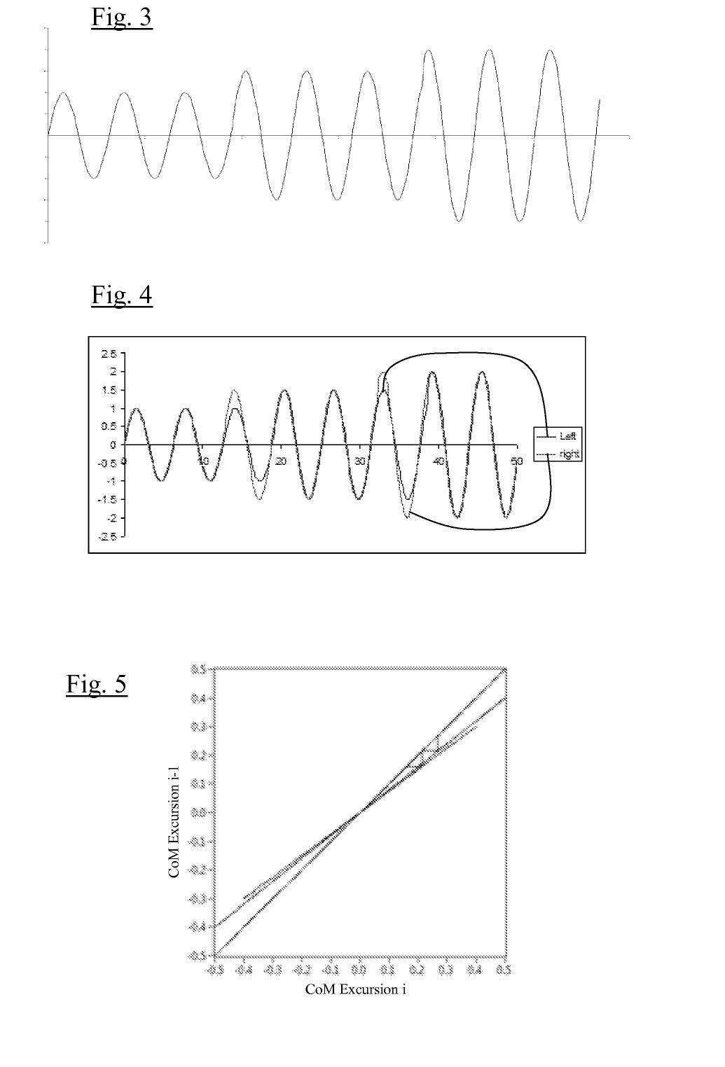

A computer generated example of a plot of CoM excursion as a function of time is shown in FIG. 3. This shows simulated results for a series of nine walking steps with CoM excursion from the starting point shown on the vertical axis and time on the horizontal axis, with the step length increasing after the first three, and the next three steps. Sample frequency in the simulation is set as 100 Hz which corresponds to the sample rate for the inertial measurement unit (IMU). This is a suitable sample frequency for gait measurement but in practice the sample rate of the IMU can vary, for example up to 120 Hz, or higher if particularly high resolution is required.

Therefore the time interval between sample height measurements is equal to 0.01seconds. The plot of FIG. 3 is over a period of 5 seconds. Therefore there are 500 sample points on the plot, and as there are nine steps there are about 55 sample points per step.

Referring to FIG. 4, in order to measure variation in gait, the computer system 22 is arranged to compare the CoM excursion for each sample point in each step with the CoM excursion at the corresponding point in the previous step. It will be appreciated that this compares the CoM excursion for a step on one leg with a corresponding CoM excursion for a step on the other leg. In order to do this, the plot of FIG. 3 is copied and shifted by one step length, resulting in two CoM excursion plots superimposed on each other as shown. Each point on one of the curves is therefore aligned with the corresponding sample point in the previous step in the other curve. Where a step is the same as the previous step, the curves are aligned, and where there is a difference between one step and the next this can be seen as a divergence between the two curves.

Referring to FIG. 5, the computer is then arranged to generate a two dimensional plot (referred to herein as a non-linear plot) of the CoM excursion for each point on one axis (the horizontal axis) against the CoM excursion for the corresponding point one step previously on a perpendicular axis (the vertical axis). For all cases where the two excursions are equal, the plot will include a point on the line at 45° through the point (0, 0). The points in the third peak and third trough where the maximum excursion of the current step is 1.5 cm and of the previous step is 1.0 cm will generate a line passing through (0, 0) and the point (1.5, 1). The points in the sixth peak and sixth trough where the maximum excursion of the current step is 2 cm and of the previous step is 1.5 cm will generate a line passing through (0, 0) and the point (2, 1.5). It will be appreciated that in this example as the two superimposed plots are in phase, because the steps are of a constant frequency, the lines on the plot of FIG. 5 all pass though the point (0, 0). However if the steps vary in period, then the original and shifted curves will not cross the horizontal axis at the same time, so a plot similar to FIG. 5 will not pass through that point.

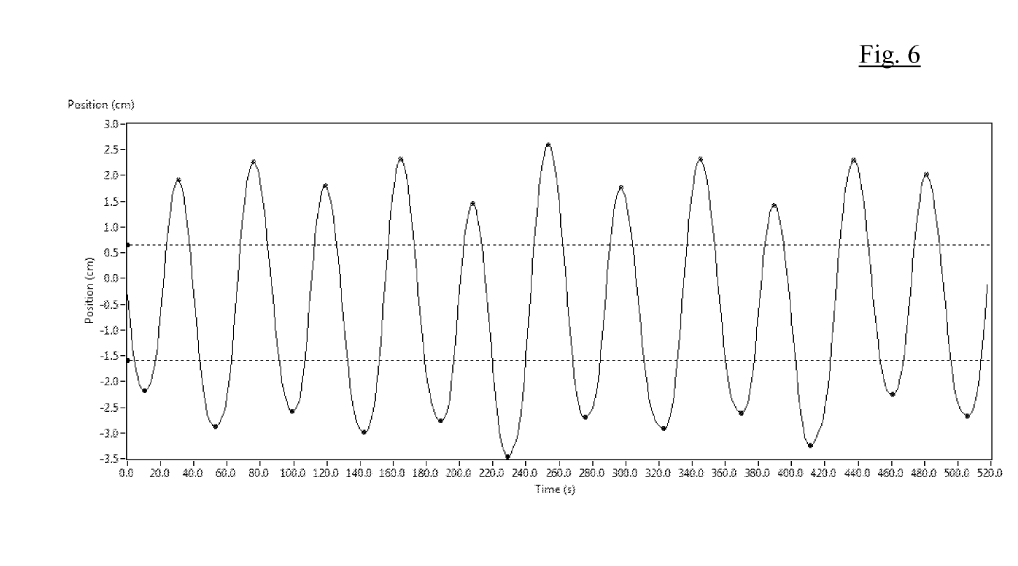

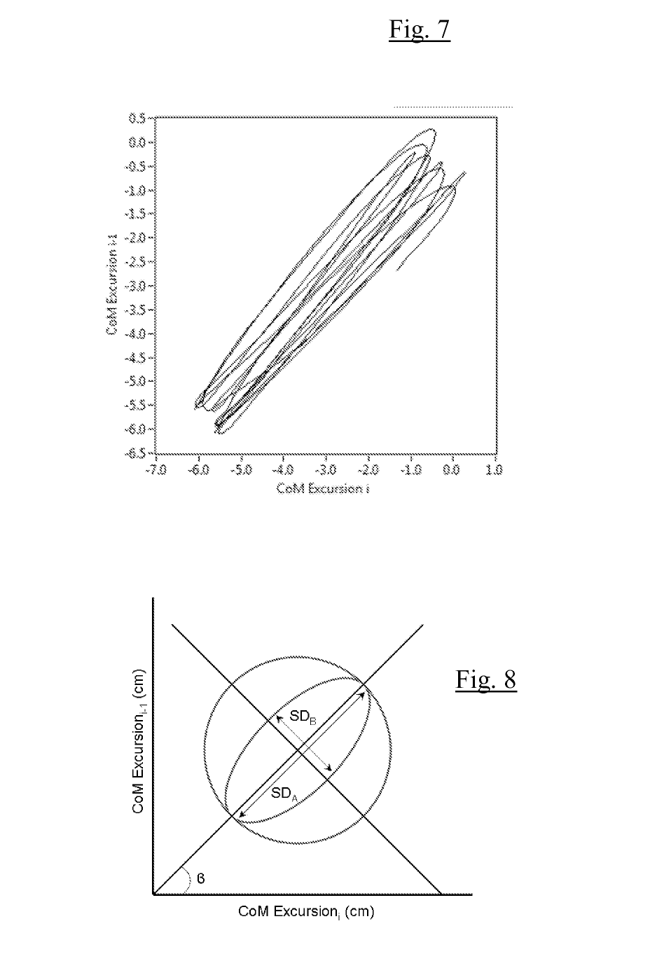

This is illustrated further with reference to FIG. 6 which shows a plot of actual measured CoM excursion as a function of sample number, with a sampling frequency of 100 Hz, for a real subject walking for 10 m. Superficially it can be seen that the oscillations in CoM excursion alternate between higher and lower amplitudes, suggesting a difference between the steps of the left and right legs, though there is obviously significant variation in the steps for each leg. Plotting the CoM excursion (CoM Excursion i) for each sample point against the CoM excursion (CoM Excursion i−1) for the sample point a fixed time (approximately equal to one step length) earlier results in a plot shown in FIG. 7. This plot shows a line joining each of the points that is produced for a respective sample time in the CoM excursion plot. The shape of the cluster of points, or the line joining them as in the plot of FIG. 7, can be analysed to determine various parameters of the plot which in turn can be used as a measure of various characteristics of the gait of the subject.

It will be appreciated that, for the shape of the cluster to be analysed in a useful way, there need to be sufficient points in the plot, and therefore sufficient sample points of measured height. In the plot of FIG. 6 there are approximately 40 sample points per step. Fewer sample points could be used, but an average of at least 10 per step is needed over the test period to be able to gather sufficient data over a relatively short test time. Also while it is simplest and desirable for the sample points to be equally spaced in time, there can be some variation in the time interval between sample points, for example to have more sample points around specific parts of the step cycle.

In some cases each pair of points may be selected on the basis of their position within the step cycle. For example if each step is considered as a sine wave and the time within each step is defined as a fraction of the whole of that step, such as in terms of an angle between 0 and 360°, then the pairs of points could be selected as those having the same relative (angular) position with in the step cycle.

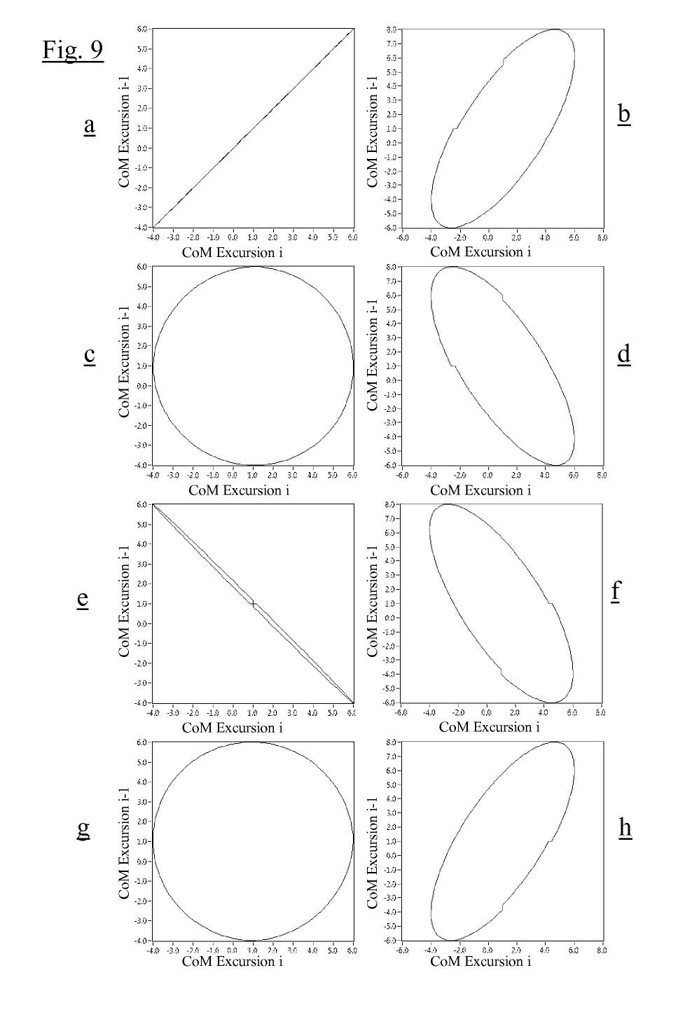

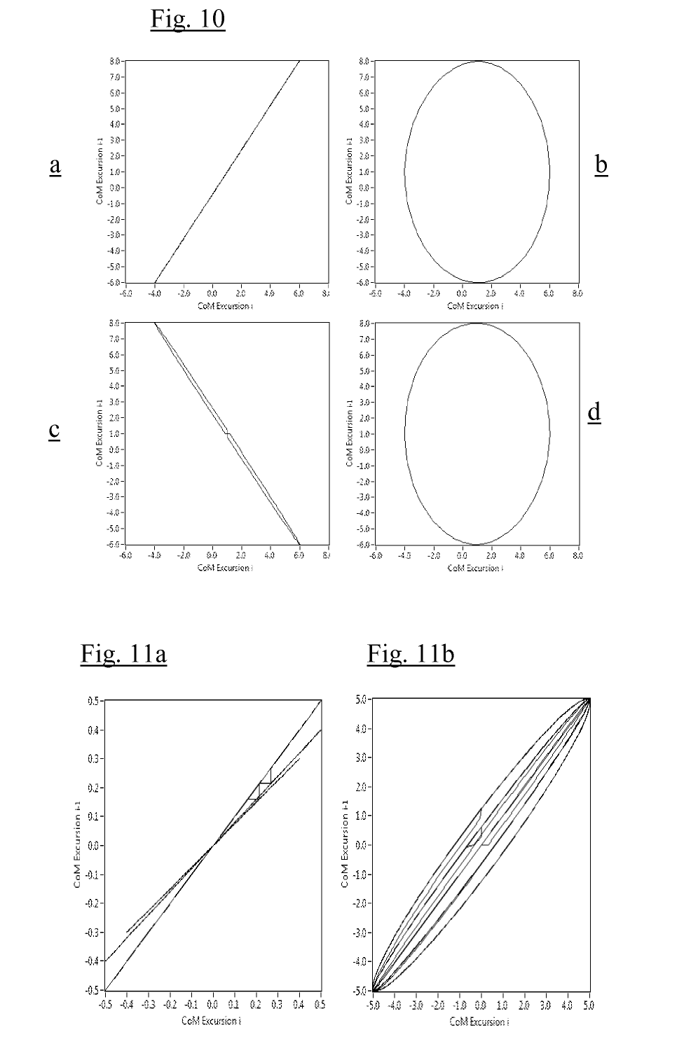

In order to better understand this analysis the theoretical exploration of these CoM plots by means of generated sine waves was performed in LabVIEW8.5 by means of a sine-generator which varied frequency, amplitude and phase shift. In order to mimic changes typically observed over a 10 metre walk the following components were altered:
1) Phase shift was generated with 45 degree steps in order to explore potential phase shifts of the vertical CoM during human walking (FIG. 9)
2) Amplitude ranges from 5 to 7 cm, which represent typical vertical CoM movement during human walking (FIG. 10)
3) Frequency values vary between 10-10.4 Hz, representing the change in walking frequency during a 10 metre walk (FIG. 11).

For each case a non-linear plot similar to that of FIGS. 5 and 7 was produced and analysed.

The analysis used for these plots will now be described with reference to FIG. 8, and it will be appreciated that the computer 20 is arranged to perform this analysis on each set of data obtained from measurement of a subject over a short walking distance, and that the parameters identified can be used by the computer system 20 to analyse the measurement data from the subject.

A least square best fit straight line is plotted against the cloud of points in the CoM excursion plot and the line is plotted as a function f=ax+b, where a is the gradient of the line and b is the intercept.

The gradient is converted to an angle β (degrees) measured from the horizontal axis in the anticlockwise direction towards the vertical axis as shown in equation (1).

β=tan⁻¹(a)·(180/π) (1)

A computer generated sine wave which assumes consistency and therefore no variability, would result in a value of β=45°.

A circle is fitted onto the data in order to find the origins of the cloud (x₀, y₀) with the radius (SD_{A}) of the given data cloud as shown in equation (2)

$${SD}_{A} = {\sum }_{i = 1}^{n} {( {( {x}_{i} - {x}_{0} )}^{2} + {( {y}_{i} - {y}_{0} )}^{2} - {r}^{2} )}^{2} \qquad ( 2 )$$

which leads to a linear equation in x₀, y₀, where (xᵢ, yᵢ) are the given points, (x₀, y₀) is the origins or midpoint and r is the unknown radius. Using the previously fitted sum of least squares the data is de-trended by subtracting the outcome of yᵢ=axᵢ+b from CoM Excursionᵢ₋₁ after which the standard deviation around the best fit straight line is calculated (SD_{B}).

An ellipse is fitted around the spread of data based on two standard deviations one SD_{A} of position measured in the direction parallel to the best fit straight line, which is the length of the ellipse, and the other SD_{B} of position measured in the direction perpendicular to the best fit straight line, which is the width of the ellipse. Ratio ∀ derived between SD_{A} and SD_{B} is determined to describe the ellipse. Furthermore angle β shows the direction of the best fit straight line through the data points indicating a level of symmetry as shown in FIG. 8.

Referring to FIG. 9, two sine waves (being theoretically non variable) generated within LabVIEW8.5 containing a frequency of 10.1 Hz, amplitude of 5 cm with an intersect of 1 cm, with zero phase shift between them, sampled at 100 Hz will result in an angle β=45° with an R² value of 1.00, SD_{A} of 4.98 cm and SD_{B} of 0. When changing the phase shift (in steps of 45 degrees) of CoMᵢ₋₁ the plots will change according to the plots shown in FIGS. 9a to 9h. Specifically FIG. 9 shows non-linear plots based on equal sine waves (frequency 10.1 Hz, amplitude 5 cm) while being out of phase by 180° (A), 225° (B) 270° (C.), 315° (D), 360° (E), 45° (F.), 90° (G) and 135° (H). It becomes clear that the sine wave produced data clouds rotate around their own axes (anti-clockwise) with change in phase shift.

CoM vertical displacement can be variable with differing limb or stride length. In order to explore the theoretical models two sign waves were generated with an amplitude of 5 cm and 7 cm respectively which represents typical human walking. The remaining configurations were similar to the previously used sine wave.

FIG. 10 shows the generated plots where it becomes visible that any variance in the CoM excursion results in a change in β. Specifically FIG. 10 shows non-linear plots based on two sine waves (frequency 10.1 Hz, amplitude 5 and 7 cm) with different while being out of phase by 180° (A), 170° (B) 360° (C.) and 90° (D).

Furthermore changes such as walking speed can be related to an increase in step length and cadence. Change in step length will result in a change of CoM vertical excursion when assuming the inverted pendulum model. The effects of the variability of step length is shown by creating three sine waves with different amplitudes (3, 5 and 7 cm respectively) representing typical vertical CoM excursions during human walking which effect is visible in FIG. 11a. Analysis with three sine waves with different frequency (10 Hz, 10.1 Hz and 10.2 Hz), in which the change (steps of 0.1 Hz) represents human gait step time or cadence variability is shown in FIG. 11b.

In use, to measure the variability of gait of a subject, the IMU 10 is strapped to the subject, and the subject walks a short distance, for example around 10 meters. During this time the IMU calculates and stores the height data for each of the sample points. The data is then input from the IMU to the computer 20 which is arranged to select pairs of sample points as described above and to generate a data set associating the two heights for each pair of sample points, for example as simple height values, or as coordinates on a plot such as the non-linear plot of FIG. 5. The computer is then arranged to control the display screen 26 to display the non-linear plot of data points, each point having a position defined by the two height values of its respective pair of sample points. The computer is also arranged to calculate the best fit straight line, circle, and oval, as described above with reference to FIG. 8, and to display those superimposed on the plot of the data points as shown in FIG. 11. The values of β, SD_{A}, SD_{B} and ∀ are also calculated and displayed on the screen. The plot itself or the numerical values calculated, or both can then be used as measures of parameters of the gait of the subject.

From the above analysis it will be clear that the computer system can be arranged to analyse the data in various different ways whilst still providing a similar measurement of gait variability. For example instead of each pair or sample points which are plotted against each other being a fixed number of samples (and hence a fixed time) apart, the data can be divided into separate steps and the first point in the left leg step plotted against the first point in the right leg step, and subsequent pairs of point plotted against each other. Alternatively the data can be separated into left leg and right leg steps, and then the left leg step data combined to form a first sine wave and the right leg data combined to form a second sine wave, and then corresponding points in the two sine waves plotted against each other. In a further alternative, rather then comparing contralateral data, i.e. data from opposite legs, ipsilateral data comparison is made in which the data for one step of one leg is compared with data for a different step of the same leg. For example the data can be divided into left and right leg steps, each of which is combined together to form left and right leg plots each being an approximate sine wave over several steps. The left leg plot is then shifted so that each left leg step is compared with the previous left leg step, and the same is done for the right leg data.

Study

Participants

Data collected from participants suffering from Parkinson's disease were analysed, and data from aged matched typical developed adults were also analysed.

Procedure

A Parkinson's Disease Questionnaire (PDQ) was administered for people with PD before partaking in this study. Participants walked over a ten-metre walkway free of obstacles at their self selected walking speed. Participants started at a static position at the zero-point and came to a complete stop at the ten-metre line. The duration of the walk was recorded by a stopwatch. An IMU was placed over the projected CoM located over the fourth lumbar vertebrae, measuring at a sample frequency of 100 Hz.

Analysis

IMU data was analysed by a program written in LabVIEW 8.5 (National Instruments,

Ireland) to obtain vertical position. Temporal and spatial gait parameters were calculated according to Zijlstra's inverted pendulum model resulting in stride length and walking speed (v_{I}). β, SD_{A}, SD_{B} and ∀ were derived by applying the non-linear method described above.

TDA and PD group were compared using an independent t-test on stride length and walking speed as well as β, SD_{A} and SD_{B}. Furthermore a Pearson's regression test was used to test for a relationship between walking speed and β for both PD and TDA. ∀ was tested by an independent t-test between PD and TDA.

Results

Descriptive measurements are displayed in Table 1.

TABLE 1
descriptive measurements showing mean for Barthel Index (BI) and
Parkinson's disease questionnaire (PDQ) for typical developed adults (TDA) and
people with Parkinson's disease (PD) where an asterisk shows a significant difference
between groups (p < 0.05). Sex is indicated as the number of males (M) taking part.

| | | | | | | Stride | | |
|---|---|---|---|---|---|---|---|---|
| | | | | Age | Diagnosis | Length | Speed | Cadence |
| Diagnosis | n | Sex | PDQ scores | (years) | (years) | (m) | (ms⁻¹) | (steps/min) |
| TDA | 10 | M = 6 | | 66.4 ± 4.4 | | 1.31 ± 0.17 | 1.36 ± 0.33* | 121.2 ± 5.6 |
| PD | 14 | M = 9 | 26 range 43-6 | 64.5 ± 6.9 | 6.3 ± 3.9 | 1.29 ± 0.21 | 1.14 ± 0.24* | 111.5 ± 011.6 |

From the theoretical exploration of this non-linear analysis it became clear that β is affected by a change in step length, SD_{A} is affected by a change in step frequency as well as step length, SD_{B} is affected by a change in step frequency and ∀ is the ratio between SD_{A} and SD_{B} defined as SD_{A}/SD_{B}

An independent t-test showed no significant difference for stride length and cadence between TDA and PD participants (p=0.615 and p=0.342). However, a difference was found for walking speed (p=0.041). Moreover an independent t-test between the TDA and PD group revealed a significant difference for β (p=0.010) and SD_{A} (p=0.004). No difference was found between groups for SD_{B} (p=0.385) and ∀ (p=0.830). Results for each group can be found in Table 2.

TABLE 2
Outcomes from non-linear method applied to gait in typical developed
adults (TDA) and Parkinson's disease (PD) showing the angle (β)
of the least square fit with SDA and SDB describing the standard
deviations of the non-linear plot with ratio ∀. An asterisk indicates
a significant difference between both groups.

| Diagnosis | β | SD_{A} | SD_{B} | ∀ |
|---|---|---|---|---|
| TDA | 42.0 ± 1.8* | 1.9 ± 0.5* | 0.4 ± 0.1 | 4.6 ± 3.9 |
| PD | 39.4 ± 3.9* | 2.5 ± 0.5* | 0.3 ± 0.1 | 7.8 ± 2.0 |

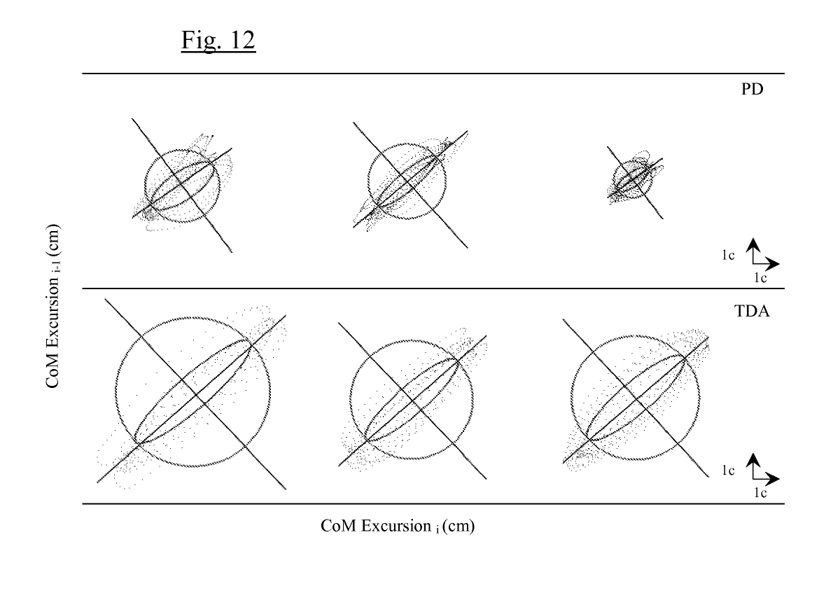

No correlation was found between β and walking speed for PD (r2=0.001 p=0.996) or TDA (r2=0.060 p=0.810). Three representative analysis figures for each condition are shown in FIG. 12.

Discussion

This study found that a non-linear analysis performed on CoM motion can be used to differentiate PD from TDA collected using IMUs over a 10 metre walk, whereas standard spatiotemporal parameters over the same distance could not. These findings are important as they promote the possibility of utilizing a non-linear variability analysis to objectively quantify gait variability and symmetry over a small sample frame, thus allowing people with PD at all stages of the disease to be monitored.

Previously, reduced step length has been reported as one of the key features of PD gait. Indeed, it has also been suggested that variability analysis may be used to closely monitor and describe gait disorders than measurements based on mean values of temporospatial walking parameters. The results of this study support this as, whilst there was no difference in step length, β and SD_{A} showed a significant difference between TDA and PD. Thus PD could be differentiated from TDA based on CoM variability (FIG. 12). The significant difference in SD_{A} may be due to increased step length variability reflected in CoM excursion. Although it should be noted that walking speed was significantly reduced for PD when compared to TDA, which has been reported previously.

Considering the novelty of this approach in exploring gait, a range of simulated CoM motions were modeled and run through the non-linear analysis in order to better understand the changes observed in PD. Group stride length variance observed in the data collected during this research, was 17 cm for TDA and 21 cm for PD during a 10 metre walk without step initiation. Assuming a constant leg length an increase in stride length of 17 and 21 cm TDA and PD would increase the vertical CoM excursion by approximately 0.3 and 0.5 cm. As shown in FIG. 11a the effect of a simulated increase in step length of these values would change the angle of the non-linear plot by 3 and 5 degrees respectively. But it is worth noticing that this diversion in angle will only occur for one gait cycle when the change in step length occurred. Once the CoM excursion is symmetric and in phase between left and right after the asymmetrical cycle, β will return to 45 degrees. Therefore it becomes visible that β is influenced by a change in step length, SD_{B} is affected by a change in step frequency, SD_{A} is affected by both a change in step length as well as step frequency and ∀ is the ratio between the change in step frequency versus step length.

As seen in FIG. 11b an increase in frequency will enlarge SD_{B} as measured over the 10 metre walk. An increase of 0.2 Hz walking frequency represents an increase of 12 steps per minute. As seen in Table 1 cadence variance is 5.6 for TDA and 11.6 for PD. A larger change in frequency results in a greater variance of SD_{B}. Perfect symmetry in lower limb stepping frequency causes the plots to assume a SD_{B} equal to zero. Indeed, with variability in CoM excursions due to step length variability in combination with step frequency variability a phase shift is expected. As shown in FIG. 9 phase shifts will change β, SD_{A} and SD_{B}. During this study, however, no phase shift was detected within either TDA or PD measurements.

Novel methods have often been used to look into gait in more depth. For example fractal dynamic analysis has been used in TDA and PD to explore stride-to-stride fluctuations. An increase in stride-to-stride variability both in stride length as step time has been observed in early and late stages of PD. Our findings are in agreement with these previous findings by showing an increase in stride length variability (SD_{A}) as well as an increase in stride-to-stride symmetry (β) within a variety of PD. Despite a visual decrease in SD_{A} and SD_{B} in FIG. 12, only SD_{A} shows a significant difference between PD and TDA. This could be explained by the reduced stride length which has also been reported by PD research. Furthermore it is noticeable that the ratio ∀ does not show a significant difference between PD and TDA. This indicates that the symmetry variability (SD_{B}) decreases by reducing stride length variability (SD_{A}).

Thus embodiments of the present invention may provide a valuable measure for clinical use. Whilst the study described above was conducted for PD this method has the potential to indicate the severity of gait impairment in PD and other populations. The present invention can therefore provide a methodology that can assist in early diagnosis in people with PD and monitor their gait during deep brain stimulation. It may also be possible to use these gait parameters to more accurately monitor efficiency of medication in PD.

1-18. (canceled)

19. A system for measuring variation in the gait of a subject while the subject takes a series of steps, the subject having a vertical position which undergoes variations during said series of steps, the system comprising a sensor arranged to measure the variations in vertical position of the subject, a processor, and a display, wherein the processor is arranged to identify a plurality of points in a first one of the steps and a plurality of points in a second one of the steps, to identify a plurality of pairs of the points, each pair comprising one point in each of said steps, to determine a value of said vertical position for each of the points in each of the pairs, and to control the display to produce a display plotting the vertical positions of the two points in each pair against each other.

20. The system according to claim 19 wherein the processor is arranged to identify the first and second steps as consecutive steps in the series.

21. The system according to claim 19 wherein the processor is arranged to identify each of said pairs of points so that the time interval between the two points in each pair is the same.

22. The system according to claim 19 wherein the processor is arranged to identify a series of points through the series of steps and to include each of the series of points in one of the pairs.

23. The system according to claim 19 wherein the processor is arranged to define a coordinate system having two axes each representing one of the vertical positions of the two points in a pair, so that the vertical positions of each pair of points can be represented by a position within the coordinate system.

24. The system according to claim 23 wherein the processor is arranged to identify the position in the coordinate system for each of the pairs of points, and to control the display so as to indicate each of those positions.

25. The system according to claim 23 wherein the processor is arranged to analyze the positions in the coordinate system associated with the pairs of points and to calculate a parameter of the positions.

26. The system according to claim 23 wherein the processor is arranged to define a line having a position which has a predetermined relationship to the positions associated with the pairs of the points, and to control the display to display the line.

27. A method of measuring variation in the gait of a subject while the subject takes a series of steps, the subject having a vertical position which undergoes variations during said series of steps, the method including measuring the variations in the vertical position of the subject, identifying a plurality of points in a first one of the steps and a plurality of points in a second one of the steps, identifying a plurality of pairs of the points, each pair comprising one point in each of the steps, determining a value of vertical position for each of the points in each of the pairs, and producing a display plotting the vertical positions of the two points in each pair against each other.

28. The method according to claim 27 wherein the first and second steps are identified as consecutive steps in the series.

29. The method according to claim 27 wherein each of the pairs of points has a time interval between them, and the pairs of points are identified so that the time interval between the two points in each pair is the same.

30. The method according to claim 27 including identifying a series of points through the series of steps and including each of the series of points in one of the pairs.

31. The method according to claim 27 further comprising defining a coordinate system having two axes each representing the vertical position of one of the two points in a pair, so that the vertical positions of each pair of points can be represented by a position within the coordinate system.

32. The method according to claim 31 comprising identifying the positions in the coordinate system for each of the pairs of points, and controlling the display so as to indicate those positions.

33. The method according to claim 31 comprising analyzing the positions in the coordinate system associated with the pairs of points and calculating a parameter of the positions.

34. The method according to claim 31 including defining a line having a predetermined relationship to the positions associated with the pairs of points, and controlling the display to display the line.

35. A system for measuring variation in the gait of a subject while the subject takes a series of steps, the subject having a vertical position which undergoes variations during said series of steps, the system comprising a memory arranged to store data recording variations in the vertical position of the subject while the subject takes the series of steps, a processor, and a display, wherein the processor is arranged to identify a plurality of points in a first one of the steps and a plurality of points in a second one of the steps, to identify a plurality of pairs of the points, each pair comprising one point in each of the steps, to determine a value of the vertical position for each of the points in each of the pairs, and to control the display to produce a display plotting the heights of the two points in each pair against each other.

36. The system according to claim 35 wherein the processor is arranged to identify the first and second steps as consecutive steps in the series.

37. The system according to claim 35 wherein the processor is arranged to identify each of said pairs of points so that the time interval between the two points in each pair is the same.

38. The system according to claim 35 wherein the processor is arranged to define a coordinate system having two axes each representing one of the vertical positions of the two points in a pair, so that the vertical positions of each pair of points can be represented by a position within the coordinate system.
